(19) United States
(12) Patent Application Publication
(10) Pub. No.: US 2026/0358347 A1
(43) Pub. Date: Nov. 12, 2026

(54) SYSTEMS AND METHODS FOR LOW-COST PROTEOME PROFILING

(71) Applicant: Brigham Young University, Provo, UT (US)

(72) Inventor: Ryan T. Kelly, Mapleton, UT (US)

(21) Appl. No.: 19/671,099

(22) Filed: May 7, 2026

Related U.S. Application Data
(60) Provisional application No. 63/803,276, filed on May 9, 2025.
(60) Provisional application No. 63/821,900, filed on Jun. 11, 2025.

(51) Int. Cl. G01N 33/68

(52) U.S. Cl. CPC G01N 33/6848

(57) ABSTRACT

According to some implementations of the disclosed systems and methods, one or more methods for performing proteome profiling are provided. In some implementations, the method includes one or more of: obtaining one or more biological samples; preparing one or more sample preparations for proteomic analysis (e.g., the sample preparation comprising protein from the biological sample); introducing the sample preparation into one or more liquid chromatography/mass spectrometry (LC-MS) workflows; acquiring mass spectrometry data; processing the mass spectrometry data to identify one or more proteome characteristics; and generating one or more proteome profiles based on the mass spectrometry data after processing. In some cases, limited amounts of material for processing can lead to decreased time and costs spent. In some cases, standards help provide reliable results despite limited operating parameters. Other implementations are also provided.

### RELATED APPLICATIONS AND PRIORITY CLAIMS

This application claims priority to United States Provisional Application No. 63/803,276, entitled “Method for low-cost proteome profiling” (Prior Attorney Docket No. 2025-030; New Attorney Docket No. 36698.8), filed May 9, 2025. This application also claims priority to United States Provisional Application No. 63/821,900, entitled “Well plate preloaded with reference standards for proteome profiling” (Previous Attorney Docket No. 2025-039; New Attorney Docket No. 36698.9), filed Jun. 11, 2025. The entire disclosure of each of the foregoing applications is incorporated herein by reference.

### FIELD

The present disclosure relates to research and diagnostic techniques, and more particularly to proteome profiling.

### BACKGROUND

Proteomics, the large-scale study and characterization of proteins within a biological sample, is a useful tool in life sciences research and clinical investigation. Proteome profiling is commonly performed using liquid chromatography in combination with mass spectrometry (LC-MS), which enables identification and quantification of thousands of proteins across a wide range of sample types. Such workflows traditionally involve multiple steps, including protein extraction, digestion into peptides, sample cleanup, chromatographic separation, mass spectrometric data acquisition, and downstream data processing and analysis. These steps traditionally require specialized instrumentation, carefully optimized parameters, and significant technical expertise to achieve reliable and reproducible results.

Despite its utility, traditional proteome profiling approaches are often resource-intensive, time-consuming, and costly. For example, per-sample costs may be relatively high due to instrument usage, consumables, reagents, and labor associated with sample preparation and data analysis. Additionally, workflows may require relatively large sample inputs and extended analysis times to achieve desired levels of proteome coverage, further increasing overall cost and complexity. As a result, widespread adoption of proteomics for high-throughput or routine applications may be limited by these practical constraints.

Thus, while techniques currently exist that are used to profile a proteome of a biological sample, challenges still exist, including those listed above. Accordingly, it would be an improvement in the art to augment or even replace current techniques with other techniques.

### SUMMARY

The present disclosure relates generally to systems and methods for proteome profiling. In particular, the present disclosure relates to workflows configured to enable efficient, scalable, and reproducible proteome profiling of biological samples, particularly where reduced sample amounts, reduced analysis times, and standardized operating conditions are desirable.

According to some implementations, a method for performing proteome profiling is provided. In some implementations, the method includes obtaining a biological sample; preparing a sample preparation for proteomic analysis from the biological sample; introducing the sample preparation into a liquid chromatography–mass spectrometry (LC-MS) workflow; acquiring mass spectrometry data; processing the mass spectrometry data to identify one or more proteome characteristics; and generating a proteome profile based on the processed data. In some implementations, the method is configured to operate across a range of biological sample types, including samples derived from one or more cell types, tissues, or biological fluids.

In some implementations, the sample preparation is configured to utilize a relatively small amount of proteinaceous material, such as picogram- to nanogram-scale amounts of protein, while still enabling identification or quantification of a substantial portion of a proteome. In some implementations, the sample preparation is generated using one or more simplified preparation workflows, including one-pot or reduced-step preparation techniques. In some implementations, the method includes selecting or limiting an amount of biological material processed to achieve a target input amount for proteomic analysis.

In some implementations, the LC-MS workflow includes one or more liquid chromatography systems and one or more mass spectrometry systems configured to analyze the sample preparation. In some implementations, the LC-MS workflow is configured to operate using relatively short chromatographic gradients or relatively rapid acquisition cycles. In some implementations, the LC-MS workflow includes one or more multi-column or parallelized configurations configured to increase throughput and reduce idle time between analyses.

In some implementations, the mass spectrometry portion of the workflow includes one or more high-resolution mass spectrometers, including Orbitrap-based systems (e.g., Orbitrap Astral) or ion mobility-enabled systems (e.g., timsTOF systems). In some implementations, the mass spectrometry data is acquired using one or more acquisition strategies, including data-independent acquisition (DIA), data-dependent acquisition (DDA), parallel accumulation–serial fragmentation (PASEF), diaPASEF, or other acquisition modes. In some implementations, the method includes using one or more standardized acquisition methods across multiple samples without substantial modification.

In some implementations, the method includes incorporating one or more standards into the workflow. For example, in some implementations, the method includes using one or more system suitability standards, external standards, or internal standards to facilitate calibration, normalization, quality control, or validation of proteomic measurements. In some implementations, the standards are used to evaluate performance of one or more portions of the workflow, including sample preparation, chromatographic separation, or mass spectrometry analysis. In some implementations, one or more of the standards are incorporated into the sample preparation, co-analyzed with the sample, or analyzed alongside the sample.

In some implementations, one or more standards are provided in a preloaded format. Indeed, in some implementations, one or more containers (e.g., wells of a multi-well plate) include preloaded standards configured to be used with one or more biological samples. In some implementations, the preloaded standards include one or more combinations of system suitability standards, external standards, and internal standards arranged across a plurality of containers. In some implementations, the preloaded format facilitates high-throughput processing of samples and reduces user preparation steps.

In some implementations, the mass spectrometry data is processed using one or more computational or analytical techniques to identify peptides, proteins, or protein groups and to determine one or more characteristics thereof. In some implementations, the processing includes one or more of spectral matching, database searching, quantification, normalization, or automated analysis techniques. In some implementations, the processing is performed using one or more automated workflows configured to reduce manual intervention and improve reproducibility.

In some implementations, the proteome profile includes one or more outputs representing proteins present in the biological sample and one or more characteristics associated therewith, including abundance values or other proteomic metrics. In some implementations, the proteome profile is used for one or more downstream applications, including comparison across samples, biomarker identification, classification of biological states, or other analytical or diagnostic purposes.

According to some implementations, a method is provided in which a plurality of biological samples are processed using a common set of operating parameters. In some implementations, the method includes preparing multiple sample preparations, introducing each sample preparation into an LC-MS workflow using the same or substantially similar operating parameters, acquiring mass spectrometry data for each sample, and generating corresponding proteome profiles. In some implementations, the use of a common set of operating parameters across multiple samples facilitates standardized and reproducible proteome profiling.

According to some implementations, a system for performing proteome profiling is provided. In some implementations, the system includes a plurality of containers preloaded with one or more standards, including a system suitability standard, an external standard, and an internal standard, and an LC-MS workflow configured to analyze contents of the containers. In some implementations, the system is configured to process a plurality of samples using a common set of operating parameters.

In some implementations, the systems and methods described herein provide improved efficiency, reproducibility, and scalability relative to conventional proteome profiling techniques. In some implementations, the systems and methods enable proteome profiling using reduced sample amounts, reduced analysis times, and standardized workflows, thereby facilitating high-throughput and cost-effective proteome profiling across a plurality of samples.

These and other features and advantages of the described systems and methods will be set forth or will become more fully apparent in the description that follows and in the appended claims.

### DESCRIPTION OF THE FIGURES

The objects and features of the present disclosure will become more fully apparent from the following description and appended claims, taken in conjunction with the accompanying figures. Understanding that these figures depict only some embodiments of the disclosed systems and methods and are, therefore, not to be considered limiting in scope, the systems and methods will be described and explained with additional specificity and detail through the use of the accompanying figures in which:

FIG. 1A shows a graphical representation of proteome coverage as a function of sample amount for different mass spectrometry platforms (lighter shading indicates additional proteins identified using Match Between Runs in DIA-NN; the left column for each sample amount corresponds with the timsTOF Ultra 2, and the right column corresponds with the Orbitrap Astral), in accordance with some embodiments of the disclosed systems and methods;

FIG. 1B shows a graphical representation of coefficients of variation as a function of sample amount for different mass spectrometry platforms (the left column for each sample amount corresponds with the timsTOF Ultra 2, and the right column corresponds with the Orbitrap Astral), in accordance with some embodiments;

FIG. 2A shows a graphical representation of linearity of peptide signal as a function of sample amount for different mass spectrometry platforms (in particular, showing the distribution of R-squared values), in accordance with some embodiments;

FIG. 2B shows a graphical representation of ion mobility distributions as a function of linearity quantiles (ion mobility vs. linearity for peptides analyzed using the timsTOF Ultra 2 mass spectrometer), in accordance with some embodiments;

FIGS. 3A–3D show graphical representations of correlation of peptide signal intensities at varying sample amounts between different mass spectrometry platforms (pairwise correlation of LFQ intensities between the Orbitrap Astral and the timsTOF Ultra 2 for 0.2, 0.5, 2, and 10 ng sample inputs), in accordance with some embodiments;

FIG. 4A shows a graphical representation of proteome coverage as a function of gradient length (in minutes) for different mass spectrometry platforms, in accordance with some embodiments;

FIG. 4B shows a graphical representation of coefficients of variation as a function of gradient length (in minutes) for different mass spectrometry platforms, in accordance with some embodiments;

FIG. 5A shows a graphical representation of overlap in identified proteins between different mass spectrometry platforms at a first gradient length, in accordance with some embodiments;

FIG. 5B shows a graphical representation of overlap in identified proteins between different mass spectrometry platforms at a second gradient length, in accordance with some embodiments;

FIG. 6A shows a graphical representation of proteome coverage as a function of sample amount for different data analysis approaches (analysis of sample inputs from 0.2 to 100 ng using a 2-column nanoLC system operated at 126 samples per day (11.4 min cycle time) and the Orbitrap Astral mass spectrometer), in accordance with some embodiments;

FIG. 6B shows a graphical representation of coefficients of variation as a function of sample amount for different data analysis approaches (as described in relation to FIG. 6A), in accordance with some embodiments;

FIG. 7 shows a schematic flow diagram illustrating a method for performing proteome profiling, in accordance with some embodiments; and

FIG. 8 shows a graphical representation of identified proteins as a function of window size when capturing mass spectrometry data, in accordance with some embodiments.

### DETAILED DESCRIPTION

A description of embodiments will now be given with reference to the figures. It is expected that the present systems and methods may take many other forms and shapes, hence the following disclosure is intended to be illustrative and not limiting, and the scope of the disclosure should be determined by reference to the appended claims.

The instant systems and methods include systems and methods for proteome profiling. Generally speaking, proteome profiling is a way to analyze a set of proteins in a cell or tissue. In some cases, proteome profiling is aimed at identifying (or identifying characteristics of) all of the proteins present in the target sample, but in some cases proteome profiling can be directed at one or more subsets of proteins. Proteome profiling can be extremely beneficial in understanding what is actively happening at a given time in a biological sample. It can be especially useful for identifying disease mechanisms, discovering biomarkers for diagnosis and prognosis, and supporting drug development by revealing how treatments affect protein expression and pathways. Because proteins carry out most cellular functions, proteome profiling provides more direct insight than genetic data alone and is valuable in areas like precision medicine, where treatments are tailored to an individual based on their particular protein profile.

Indeed, proteomics, the large-scale study of proteins, is an indispensable field in the realm of life sciences. Proteins are the dynamic workhorses of the cell, playing critical roles in virtually every biological process. Unlike the genome, which remains relatively constant, the proteome is highly dynamic, changing in response to various stimuli, environmental conditions, and developmental stages (in some cases, along a relatively short timeline, such as on the order of days, hours, minutes, or even seconds). By analyzing the proteome, scientists can gain deep insights into the functional mechanisms of cells, tissues, and organisms. This can have significant implications for understanding disease pathways, identifying potential biomarkers for early diagnosis, and discovering new therapeutic targets. Proteomics also aids in the development of personalized medicine, where treatments can be tailored to the unique proteomic profile of an individual. In essence, proteomics bridges the gap between the static genome and the dynamic biological processes, offering a comprehensive view of the molecular underpinnings of life.

Although competing methods of proteome profiling exist, many such methods are time-consuming, difficult to implement, and costly. Indeed, in some cases, the systems and methods described herein can accomplish proteome profiling at a greatly decreased cost (e.g., at least 5-fold, 10-fold, 15-fold, 20-fold, or more of a reduction in cost over many competing methods). In contrast to genome sequencing, which has been commoditized and which has seen a ~100,000-fold reduction in cost per genome this century, proteome profiling according to competing methods typically remains very expensive and has seen virtually no cost reduction over the same timeframe. A survey of prices at core facilities may indicate that a single liquid chromatography/mass spectrometry analysis of a biological sample can cost $200 to more than $1,000, depending on the facility, whether or not the customer is academic or industry, or other factors. The high cost may be due to a number of different factors, such as: (a) little standardization in a field with a diversity of sample types, data acquisition settings, separation techniques, and data analysis workflows; (b) large sample sizes (conventional sample sizes often contain hundreds of nanograms up to micrograms or even milligrams of biological material, which must undergo extensive sample preparation to remove lipids and insoluble debris, shear the DNA, and otherwise be ready for protein analysis; failure to perform these slow and expensive steps may result in a liquid chromatography column becoming clogged, or in contamination of a mass spectrometer, leading to the need for frequent instrument maintenance and repairs); (c) use of outdated technology (for example, until recently, mass spectrometers were much slower and less sensitive, which led to a steep tradeoff between the amount of sample analyzed and the number of proteins identified; that is, more biological material typically led to more proteins being quantified; in addition, a steep tradeoff existed between analysis times and the number of proteins identified, with long analysis times required to quantify more proteins); and (d) lack of consistent data analysis workflows across different research groups, leading to difficult-to-reproduce results and analyses.

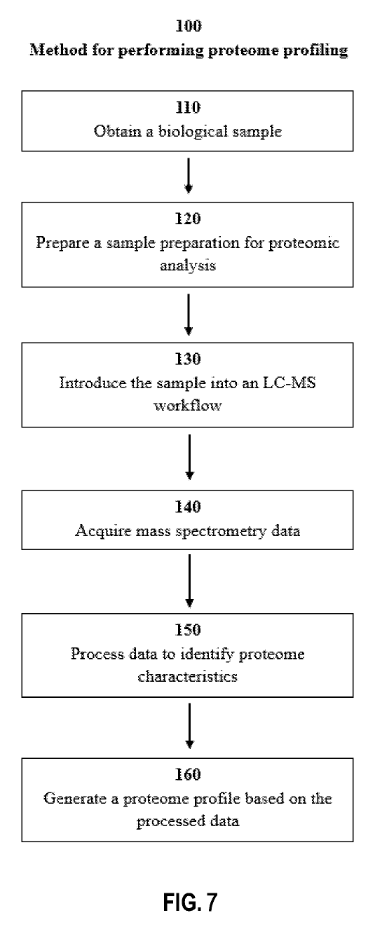

According to some embodiments of the systems and methods described herein, one or more methods 100 for performing proteome profiling are provided. For example, as illustrated by FIG. 7, some embodiments of the method include one or more of: obtaining one or more biological samples (as shown by box 110); preparing one or more sample preparations for proteomic analysis (as shown by box 120); introducing the sample into one or more LC-MS workflows (as shown by box 130); acquiring mass spectrometry data (as shown by box 140); processing the data to identify proteome characteristics (as shown by box 150); and generating one or more proteome profiles based on the processed data (as shown by box 160).

As mentioned above, some embodiments of the method 100 include obtaining one or more biological samples (e.g., as shown by box 110 of FIG. 7). In this regard, any suitable proteinaceous biological sample can be used, including one or more blood samples, urine samples, hair samples, skin samples, saliva samples, plasma samples, serum samples, cerebrospinal fluid (CSF) samples, tissue samples, biopsy samples, cell culture samples, cell samples, microbial samples, environmental samples, exosome samples, archaeological samples, other protein-containing biological materials, or any other suitable samples. Importantly, some embodiments of the biological sample include protein from multiple different cells or types of cells (e.g., any combination of the different biological samples described above) so that the resulting proteomic analysis is representative of more than just one cell or cell type or organism (using multiple types of cells from a single individual can help obtain a more complete picture of that individual’s proteome, while using multiple cells from different individuals can help obtain a more complete picture of an average proteome across individuals). The biological sample can be obtained in any suitable manner, such as through blood draw, urine collection, hair collection, cheek swab, tissue biopsy, surgical resection, needle aspiration, cell harvesting, microdissection, obtaining from a sample bank, obtaining from a previously-collected sample, obtaining from a biobank or repository, collected from the environment, or any other suitable collection or isolation techniques.

In some cases, notwithstanding that some embodiments utilize biological material from multiple parts of a patient (e.g., various combinations of the types of biological samples described above), some embodiments of the biological sample are limited in quantity to allow for faster preparation and processing, as discussed in more detail below. In some cases, this allows for easier sample collection. For example, in some embodiments, only a finger-prick or other minimally invasive technique is required for blood collection, while competing methods may require filling one or more tubes or vials of blood using an IV catheter or similar device. In some embodiments, no invasive collection is required, as suitable quantities of protein (for use in connection with the instant systems and methods) can be obtained through non-invasive collection methods.

As mentioned above, some embodiments of the method 100 include preparing one or more sample preparations for proteomic analysis (e.g., as shown by box 120 of FIG. 7). In some embodiments, preparing the sample preparation includes processing a biological sample to produce one or more protein-derived analytes suitable for LC-MS analysis, such as peptides generated via digestion of proteins. The preparation can be done in any suitable manner, such as by utilizing one or more of lysis, denaturation, reduction, alkylation, digestion, or dilution techniques. In some cases, the sample preparation includes one or more simplified workflows, such as one-pot preparation techniques, which can be performed with minimal handling, and in some cases without conventional complicated cleanup procedures. By way of non-limiting illustration, FIG. 7 shows some embodiments in which a sample preparation is generated prior to introduction into an LC-MS workflow (e.g., as shown by boxes 120 and 130).

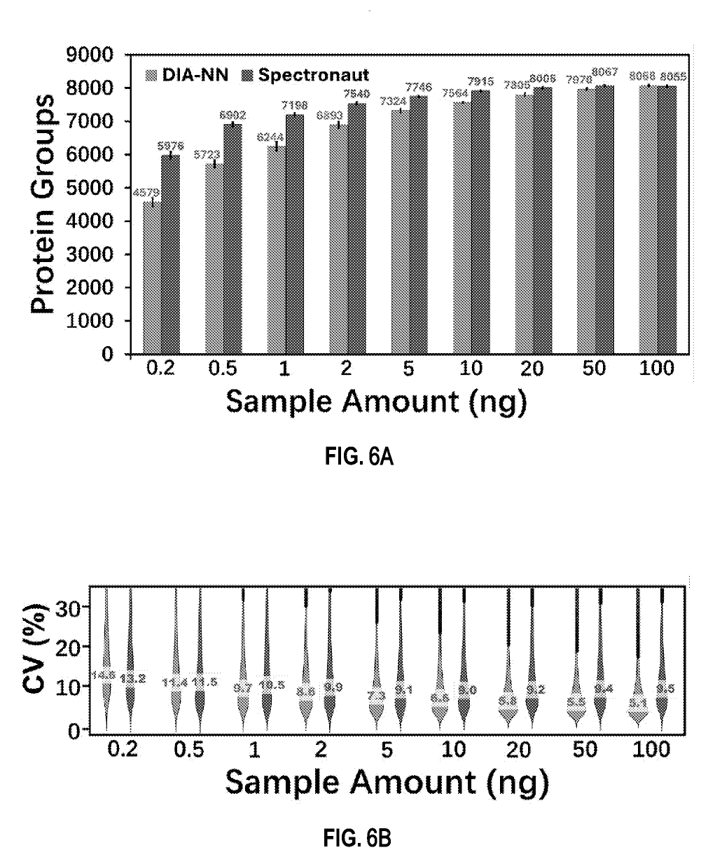

In traditional proteomics, a large amount of proteinaceous material is typically used, such as at least 1 µg, and up to hundreds of micrograms or even milligrams of protein being required for satisfactory results to be obtained. However, according to some embodiments of the systems and methods of the present disclosure, the sample preparation is configured to utilize a relatively small amount of starting material (e.g., on the order of picograms to nanograms of protein), while still enabling identification or quantification of the proteome. Indeed, in some embodiments, preparing the sample preparation includes selecting or limiting an amount of the biological sample to be processed, such that a target amount of protein is prepared for analysis that is less than about 100 pg to 250 ng (or any suitable subrange thereof or amount therein, such as less than about 100 ng, less than about 50 ng, less than about 10 ng, or any other suitable amount or subrange thereof). For example, according to some embodiments, effective proteome profiling is obtainable with about 200 pg to 100 ng of proteinaceous material. In some cases, the low-nanogram range (e.g., about 1 ng, about 5 ng, about 10 ng, about 20 ng, about 30 ng, about 50 ng, about 75 ng, about 100 ng, or any other suitable low-nanogram amount) may be ideal for minimizing the costs compared to the benefit derived in the proteome profiling. Accordingly, in some cases, the method includes using a protein sample size of about 50ng (or any other suitable amount within the ranges described above) as a standard protocol across separate proteome profiling cases, such that each case can be swiftly performed as part of a streamlined procedure. That said, in some cases, the systems and methods described herein enable satisfactory results to be obtained with even picograms of protein (e.g., about 100 – 1000 pg, 250 – 500 pg, or any other suitable amount). In some cases, an amount is chosen so as to allow use of a simple and inexpensive one-pot preparation, yet to still be large enough to nearly saturate the amount of biological information obtained from the sample using the method 100. As shown in FIGS. 6A–6B, in accordance with some embodiments, this “sweet spot” is in the range of 10–100 ng for at least some preparations.

In some embodiments, a sufficiently small amount of the biological sample is used to ensure compatibility with a one-pot preparation technique. In this regard, the one-pot preparation technique can include any suitable technique that allows for preparation and analysis of the sample in a single container (e.g., a well of a well plate, a tube, a vial, or another sample container). For example, in some cases, mass spectrometry-compatible reagents are added to the same container of the sample, and the resulting mixture is analyzed directly (without the need for additional mixing, reacting, or processing containers or chambers that would require subsequent cleanup). In most cases, 200 ng or less of protein is usable with the one-pot preparation technique (which, fortuitously, is also sufficient material to saturate or nearly saturate the number of proteins identified by the mass spectrometer, in some embodiments), but any suitable amount of protein (e.g., as discussed in the preceding paragraph) can be used.

As mentioned above, some embodiments of the one-pot preparation technique include introducing one or more MS-compatible reagents. Although any suitable MS-compatible reagents can be used, some embodiments include adding one or more of: solvents (e.g., water, acetonitrile, methanol, ethanol, isopropanol, propanol, tetrahydrofuran, dichloromethane, nitromethane, toluene, acetone, or any other suitable MS-compatible solvents); ionization enhancers (e.g., formic acid, acetic acid, trifluoroacetic acid, heptafluorobutyric acid, or any other suitable ionization enhancers); buffers (e.g., ammonium acetate, ammonium formate, ammonium bicarbonate, ammonium hydroxide, or any other suitable buffers); protein or peptide preparation reagents (e.g., trypsin, Lys-C, chymotrypsin, Glu-C, other proteases, reducing agents, alkylating agents, surfactants, urea, labeling agents, or any other suitable preparation agents).

In some embodiments, the one-pot preparation technique includes incubating the container (e.g., sealing the container to minimize evaporation, allowing preparation agents time to react with or otherwise prepare the proteins, mixing the container, or otherwise incubating the sample preparation).

In some embodiments, the method 100 includes filtering the biological material prior to preparing it, or otherwise selectively adding protein-heavy material to the one-pot preparation such that there is not a substantial amount of insoluble debris, lipids, or other contaminants that might require loss-prone, costly, or labor-intensive offline cleanup steps.

In some embodiments, preparing the sample preparation includes incorporating one or more standards (e.g., preparations configured to be used as controls, or to otherwise facilitate calibration, normalization, quality control, or validation of proteomic measurements) into the sample preparation, or using one or more standards alongside or otherwise in connection with the sample preparation. For example, in some embodiments, the standards include one or more system suitability standards, external standards, internal standards, or any other standards configured to ensure that the proteome profiling method performs to a suitable quality standard.

Although the different types of standards (the system suitability standards, external standards, and internal standards) can play different roles (as discussed below), the standards generally can include preparations that have one or more proteins, peptides, labeled peptides, isotopically labeled compounds, or other reference materials having known identities, concentrations, or other response characteristics. In some embodiments, the standards are configured to be co-analyzed with the prepared samples during one or more portions of the LC-MS analysis, thereby enabling comparison of measured signals to known reference values. In some embodiments, the use of such standards facilitates reproducibility across runs, comparability across samples, or consistency across different instruments or workflows, which is (in some cases) especially important when dealing with streamlined workflows that operate using relatively small amounts of biological material, relatively short uses of costly equipment, or otherwise in accordance with the systems and methods described herein in manners that allow for reduction of cost, complexity, or other undesirable characteristics of proteome profiling. In some embodiments, one or more standards are used separate from the samples of interest, such as by being analyzed periodically in a similar manner to the samples of interest as an additional quality check (e.g., every other run, every five runs, every ten runs, every twenty runs, every fifty runs, or at any other suitable interval).

In some embodiments, the system suitability standard, the external standard, and the internal standard are used in combination within a single workflow such that (i) instrument performance, (ii) sample preparation performance, and (iii) quantitative normalization are each independently validated within the same batch of samples and across batches.

Where the systems or methods include one or more system suitability standards, the system suitability standards can include any standards configured to provide controls or information regarding the performance of the liquid chromatography separation or the mass spectrometry analysis. For example, in some cases, the system suitability standards include samples of known composition that are similar in complexity and amount (or other characteristics) to the sample of interest. In some embodiments, the system suitability standards are provided ready-to-use such that they require no additional sample preparation for the performance of proteome profiling in any particular case. For example, some embodiments of the system suitability standards are pre-aliquoted or preloaded into one or more containers (e.g., wells of a multi-well plate), such that they can be used directly without user preparation (as discussed in more detail below).

Although other types of system suitability standards may be used, in some cases, some embodiments of the system suitability standards include one or more multi-species proteomes having distinct ratios. Indeed, the system suitability standards can include any suitable known proteome mixture, such as a mixture of any known percentages of various organisms (e.g., human, E. coli, yeast, plant, animal, bacterial, fungal, or other proteinaceous matter), or any known peptide reference mix (e.g., Promega® 6x5 peptide mixture, Thermo® 7x5 peptide mixture, or any other reference mix). As examples, in some cases, the system suitability standard includes a 65% protein digest from a human cell line, 30% yeast peptides, and 5% E. coli peptides. In some cases, the system suitability standard includes a 65% protein digest from a human cell line, 15% yeast peptides, and 20% E. coli peptides. In some cases, the system suitability standard includes a Promega® 6x5 peptide mixture. In some cases, the system suitability standard includes a Thermo® 7x5 peptide mixture.

In some embodiments, multiple sets (e.g., 2, 3, 4, 5, or any other number) of system suitability standards are used. For example, in some cases, a first set of system suitability standards includes a 65-30-5 mix of human-yeast-E. coli proteins, and a second set of system suitability standards includes a 65-15-20 mix of human-yeast-E. coli proteins. In some cases, the method includes alternating between the multiple sets at regular intervals (e.g., between 1-50 runs, such as every run, every 2 runs, every 5 runs, every 10 runs, every 15 runs, every 20 runs, or at any other suitable interval), which can provide distinct advantages. Some such advantages may include: protein coverage for all the species represented in the mixture combined, as well as for each species separately; peak widths and shapes for a wide variety of peptides, or specific target peptides; peak intensities for known peptides; mass-to-charge ratios of specific precursors to determine mass accuracy; mass-to-charge ratios and intensities of specific fragments to determine fragmentation efficiencies; quantitative accuracy as determined by comparing the actual ratios to the known ratios of the multi-species peptides measured in the mixtures; absolute lower limits of detection; retention time alignment as determined by evaluating known peptides across the gradient; or other benefits. Thus, some embodiments include alternating between different system suitability standards every 10–20 runs, or at any other suitable interval.

In some embodiments, the method 100 includes analyzing the system suitability standards prior to, during, or after analysis of the biological sample to assess performance of one or more components of the workflow. For example, some embodiments of the system suitability standards are configured to provide information relating to one or more of: chromatographic performance (e.g., retention time stability, peak shape, separation efficiency, or other performance characteristics); mass spectrometry performance (e.g., mass accuracy, sensitivity, signal intensity, dynamic range, ion transmission, or other performance characteristics); overall system reproducibility; or other aspects of the overall LC-MS workflow. Thus, the system suitability standards can be used to determine whether the system is operating within one or more predefined performance thresholds prior to, during, or after analysis of one or more biological samples. In some embodiments, the system suitability standards are used to enable consistent operation of the proteome profiling workflow across multiple runs, instruments, or laboratories, especially in workflows utilizing relatively low sample input amounts.

According to some embodiments, the method 100 includes using the system suitability standards to determine whether one or more runs should be accepted, repeated, or discarded, including by comparing measured performance characteristics to one or more predefined acceptance criteria. For example, the system suitability standards can be used to determine whether the liquid chromatography system or the mass spectrometer is performing sub-optimally. In this regard, shifting retention times or asymmetric peaks may indicate a challenge with the liquid chromatography system, while shifting masses or low intensity precursors or fragments may indicate a challenge with the mass spectrometer. Inaccurate A/B ratios (e.g., when using multiple sets of system suitability standards) may indicate a problem with quantification.

Where the systems or methods include one or more external standards, any suitable standards that enable the effectiveness of the sample preparation to be determined can be used. For example, in some cases, external standards include samples of known composition that contain proteins that need to be prepared, such as a cell lysate, or standard peptides having one or more cleavage sites in their amino acid sequences, that have not yet been digested. In some embodiments, the method 100 includes preparing the external standards alongside the samples of interest (including digesting them with protease, or performing any other suitable preparation protocol), such that if there is an error in preparation, the external standards will not yield the expected results, indicating that a subsequent preparation should be performed.

Although any suitable external standard that allows for the effectiveness of the preparation to be determined can be used, some embodiments include one or more undigested cell lysates having known characteristics (e.g., the lysate is provided in one or more set concentrations and amounts, and the protein content has previously been evaluated and recorded, such that certain values are expected to be attained through proper preparation and processing). In some embodiments, the external standard does not include (i.e., it lacks) pre-processed peptides (e.g., a 6x5 or 7x5 mixture, or other processed peptides, such as those used for system suitability standards), so that the effectiveness of the external standard is dependent on proper preparation.

For clarity, some embodiments of the external standards are configured to provide one or more expected outputs following preparation, such that effectiveness of the preparation can be evaluated based on comparison between measured results and expected values. For example, in some embodiments, the external standards are configured to produce one or more known peptides, peptide abundances, peptide ratios, or other proteomic characteristics following proper preparation. In some cases, deviations between the expected and observed outputs are used to evaluate one or more aspects of preparation performance, including digestion efficiency, sample recovery, reproducibility, or consistency across one or more samples or batches. In some embodiments, the external standards are used to evaluate preparation performance across multiple samples prepared in parallel, such that systematic errors affecting a plurality of samples can be identified. Thus, the external standards can be used to determine whether one or more preparation steps should be repeated, adjusted, or validated prior to downstream analysis.

Where the systems or methods include one or more internal standards, the internal standards can include any suitable standards that are configured to be incorporated into the sample preparations themselves, such that they are analyzed together with the analytes of interest during LC-MS analysis. For example, in some embodiments, the internal standards include one or more peptides, labeled peptides, isotopically labeled compounds, or other reference materials present at known concentrations that are introduced into the sample preparation prior to analysis. In some embodiments, the internal standards are configured to enable normalization of signal intensities across one or more samples, including by comparing measured signal intensities of analytes of interest to signal intensities of the internal standards. Thus, in some cases, the internal standards facilitate retention time alignment, signal intensity or quantitative normalization, determination of detection limits, or compensation for variation in instrument performance across runs.

Although the internal standards can include any components configured to be spiked into the samples themselves for co-processing, some embodiments of the internal standards include one or more mixtures containing a relatively small number of different peptides having known concentrations that are selected such that they do not substantially interfere with detection of analytes of interest, while still providing sufficient signal for normalization and calibration purposes. For example, in some embodiments, the internal standards include one or more peptide mixtures (e.g., 6×5 or 7×5 peptide mixtures, optionally supplemented with additional peptide reference standards (e.g., Thermo® 15-peptide PRTC mixture, or any other suitable reference mixture)). In some embodiments, the ratio of signal from the internal standards to signal from sample-derived peptides is used to normalize data across multiple runs, including in cases where instrument sensitivity varies over time or across different instruments (which can be very important for achieving useful results, particularly when small sample sizes are used with limited instrumental measurements).

According to some embodiments, the internal standards include sufficiently few types of peptides that the peptides do not substantially mask the signal from the sample peptides, but enough peptides to facilitate chromatographic alignment and intensity normalization.

In some embodiments, one or more of the standards (whether system suitability standards, external standards, internal standards, or otherwise) are provided in a preloaded format. For example, in some embodiments, one or more containers (e.g., wells of a well plate, vials, cartridges, or other sample holders) include one or more preloaded standards prior to loading of the biological sample (with the standards being configured to be used with or alongside the biological sample, as appropriate). In some embodiments, the preloaded standards include one or more combinations of internal standards, external standards, and system suitability standards arranged in a predetermined manner across a plurality of containers. Such a preloaded format can facilitate high-throughput processing of multiple samples, including by enabling consistent preparation across a plurality of samples with minimal user intervention.

In some embodiments, the preloaded standards are arranged to support one or more standardized workflows. For example, in some embodiments, one or more multi-well plates include a plurality of wells, each well having its own designated use. For example, one or more wells may include one or more standards configured for use in proteome profiling, and one or more wells (in some cases, the same wells as the standards, and in some cases, different wells) are configured to receive a biological sample. In some embodiments, different wells include different standards or different concentrations of standards, thereby enabling calibration curves, quality control checks, or performance validation across a batch of samples. For example, in some cases, one well includes a first concentration of a first type of standard (e.g., system suitability standards, external standards, or internal standards), and another well includes a second concentration of the first type of standard (e.g., system suitability standards, external standards, or internal standards).

In some embodiments, one or more components of the standards or sample preparation reagents are included in the containers in a dried or stabilized form. For example, in some embodiments, one or more wells include preloaded internal standards, external standards, or system suitability standards together with one or more reagents configured to facilitate sample preparation, such as surfactants, buffers, proteases, denaturants, reducing agents, alkylating agents, or other components. In some embodiments, the preloaded materials are dried or otherwise stabilized (e.g., via lyophilization, vacuum drying, or other processing) and stored for later use, such that a user can add a biological sample and/or buffer to reconstitute the materials prior to analysis. In some cases, the method 100 includes storing the prepared wells at freezing temperatures (such as between -20° C. and -80° C. or any other suitable temperature as may be desirable for extended preservation of the standards). Relatedly, some embodiments include reconstituting the standards (e.g., by thawing and/or adding one or more samples, surfactants, buffers, proteases, or other components) prior to performing LC-MS analysis.

In some embodiments, different wells include different combinations of preloaded materials thereby enabling flexible workflow design and minimizing user preparation steps. For example, in some cases, a well plate is provided with a first well pre-loaded with a first standard and a second well pre-loaded with a second standard (and any number of additional wells pre-loaded with any number of additional standards, such as a third well with a third standard, a fourth well with a fourth standard, a fifth well with a fifth standard, etc.). For example, in some cases, a well plate includes a first well having one or more system suitability standards, a second well having one or more external standards, a third well having one or more internal standards (and having enough additional space as needed to receive a sample), and a fourth well including one or more system suitability standards, external standards, and internal standards combined together.

In some embodiments, one or more of the standards are used in connection with automated data analysis workflows. For example, in some embodiments, software is configured to analyze signals obtained from one or more of the standards and to normalize one or more analyte signals based on the standard signals (e.g., automatically, manually, or otherwise). In some embodiments, such automated workflows include normalization, calibration, quality control evaluation, identification of outlier samples, drift correction, batch normalization, recalibration, or other method components. In some embodiments, incorporation of standards into the workflow facilitates automated processing and reduces manual intervention in proteome profiling analysis. As mentioned above, some embodiments of the standards are configured to assess one or more aspects of system performance, including instrument sensitivity, chromatographic separation, mass accuracy, quantitative reproducibility, or other characteristics of the proteome profiling process. Thus, the use of preloaded standards and standardized plate configurations can reduce variability between samples, simplify workflow design, and enable consistent, scalable proteome profiling across a large number of samples and in different environments—especially where small sample sizes, limited-time mass spectrometry runs, or other endeavors are taken to reduce costs. Indeed, in some embodiments, proper use of the different types of standards described herein leads to the surprising result of enabling proteome profiling using the limited sample sizes and other cost-saving parameters described herein.

As shown by boxes 130-140 of FIG. 7, some embodiments include introducing the sample (and any applicable standards, as discussed above) into an LC-MS workflow and acquiring mass spectrometry data. In this regard, any suitable LC-MS workflow can be used to analyze the prepared sample. For example, in some embodiments, introducing the sample includes loading the sample preparation onto one or more chromatographic columns, separating one or more components of the sample via liquid chromatography, and subsequently introducing the separated components into a mass spectrometer for analysis.

The liquid chromatography (or substitute, such as any suitable separation technique) portion of the LC-MS workflow can include any suitable liquid chromatography device or technique, any suitable electrophoresis technique or device, or any suitable substitute. For example, some embodiments include one or more nano-flow liquid chromatography systems, micro-flow systems, capillary-flow systems, high-performance liquid chromatography (HPLC) systems, ultra-high-performance liquid chromatography (UHPLC) systems, or other chromatographic configurations. In some embodiments, the liquid chromatography system includes one or more: columns (e.g., packed columns, monolithic columns, capillary columns, narrow-bore columns, columns having integrated emitters, or other columns); pumps; valves; sample injection systems (e.g., trap-and-elute configurations, direct injection configurations, or other sample introduction techniques).

In some embodiments, the liquid chromatography portion is configured to operate at one or more flow rates, including nano-flow rates (e.g., less than about 1 µL/min), micro-flow rates (e.g., between about 1 µL/min and about 100 µL/min), or any other suitable flow rates. In some embodiments, the liquid chromatography portion includes one or more chromatographic gradients (e.g., solvent gradients, step gradients, linear gradients, nonlinear gradients, or other gradient profiles) configured to separate peptides or other analytes over a selected time period. In some embodiments, the chromatographic gradient is configured to operate over a relatively short duration (e.g., less than about 60 minutes, less than about 30 minutes, less than about 20 minutes, less than about 10 minutes, less than about 5 minutes, less than about 3 minutes, or any other suitable duration or subrange thereof), although longer durations may also be used.

In some embodiments, the liquid chromatography portion includes one or more multi-column systems, parallelized column configurations, or column-switching arrangements configured to increase throughput or reduce idle time between analyses. For example, in some embodiments, a plurality of columns are operated such that one column is used for sample loading while another column is used for analyte separation, thereby increasing overall duty cycle. In some embodiments, the liquid chromatography system is configured to achieve a high duty cycle (e.g., greater than about 50%, greater than about 75%, greater than about 90%, or approximately 100%), thereby facilitating analysis of a large number of samples over a given period of time.

The mass spectrometry portion of the LC-MS workflow can include any suitable mass spectrometer or mass spectrometry technique, or any suitable substitute. For example, some embodiments include one or more high-resolution mass spectrometers, time-of-flight mass spectrometers, Orbitrap-based mass spectrometers, ion mobility-enabled mass spectrometers, quadrupole mass spectrometers, hybrid mass spectrometers, or other mass spectrometry systems. In some embodiments, the mass spectrometry portion includes one or more mass spectrometers selected from commercially available systems, such as the Thermo Fisher Orbitrap Astral, Orbitrap Astral Zoom, other Orbitrap-based instruments, or Bruker timsTOF systems (e.g., timsTOF Ultra, timsTOF Ultra 2, timsTOF SCP, timsUltra AIP, or other ion mobility-enabled systems), although any suitable mass spectrometer can be used. In some embodiments, the mass spectrometry system includes one or more ion sources configured to ionize for analysis the samples (and standards) provided (e.g., electrospray ionization sources), ion optics, mass analyzers, detectors, or other components configured to facilitate analysis of peptide or protein-derived analytes.

In some embodiments, the method 100 includes operating the mass spectrometer in accordance with one or more particular acquisition strategies. For example, in some embodiments, the mass spectrometry system is operated to perform data-independent acquisition (DIA), data-dependent acquisition (DDA), parallel accumulation–serial fragmentation (PASEF), diaPASEF, parallel reaction monitoring, selected reaction monitoring, multiple reaction monitoring, or other acquisition models. In some embodiments, the mass spectrometry portion includes one or more acquisition methods configured to collect precursor ion data, fragment ion data, or both, across one or more mass-to-charge (m/z) ranges. In this regard, the acquisition method can include one or more isolation windows, scan cycles, ion accumulation times, ion mobilities, fragmentation energies, high-field asymmetric ion mobility (FAIMS) settings, or other operation parameters configured to generate proteomic data from the sample. In some embodiments, the acquisition method is configured to analyze a plurality of peptides within a single cycle, thereby facilitating comprehensive proteome profiling.

In some embodiments, the mass spectrometry portion is configured to operate using one or more standardized acquisition methods. For example, in some embodiments, a predefined set of acquisition parameters (e.g., m/z ranges, scan speeds, isolation window widths, ion mobility settings, fragmentation parameters, or other acquisition settings) is used across a plurality of samples without substantial modification. In some embodiments, the use of such standardized acquisition methods reduces or eliminates the need for per-sample or per-experiment method optimization. In some embodiments, the mass spectrometry portion is configured such that a single acquisition method, or a limited number of acquisition methods, can be applied across a range of sample input amounts, sample types, or experimental conditions, thereby facilitating streamlined and scalable proteome profiling workflows. While this would not be effective if used in accordance with some competing systems and methods, performance in accordance with the systems and methods described herein (e.g., as used in connection with the sample sizes, preparation processes, standards, and other system and methods features presented in this disclosure) surprisingly enables streamlined (and faster) data acquisition parameters of the mass spectrometry system.

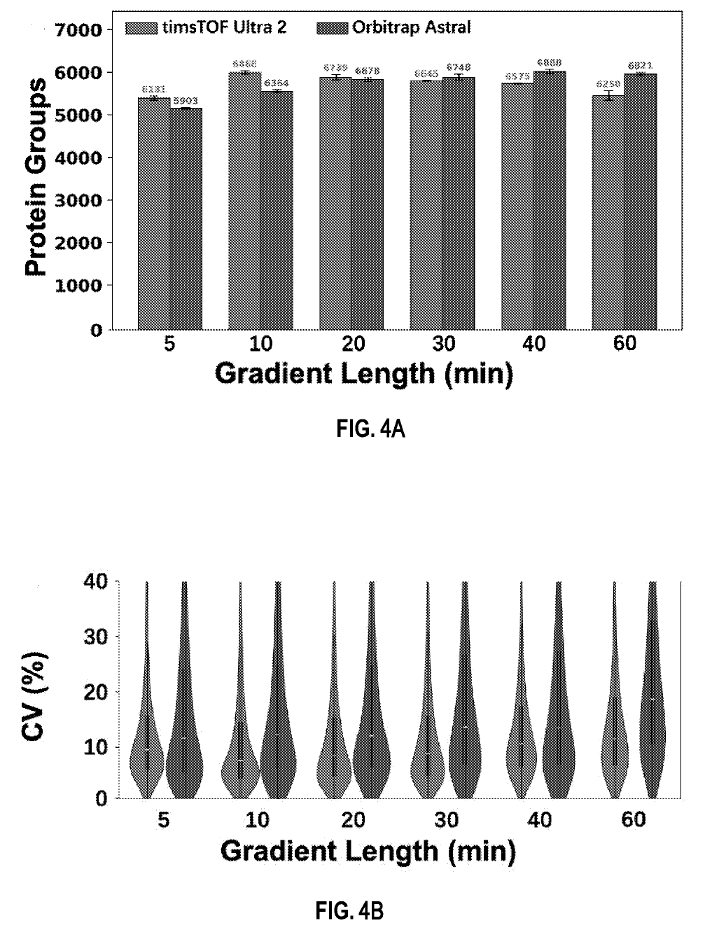

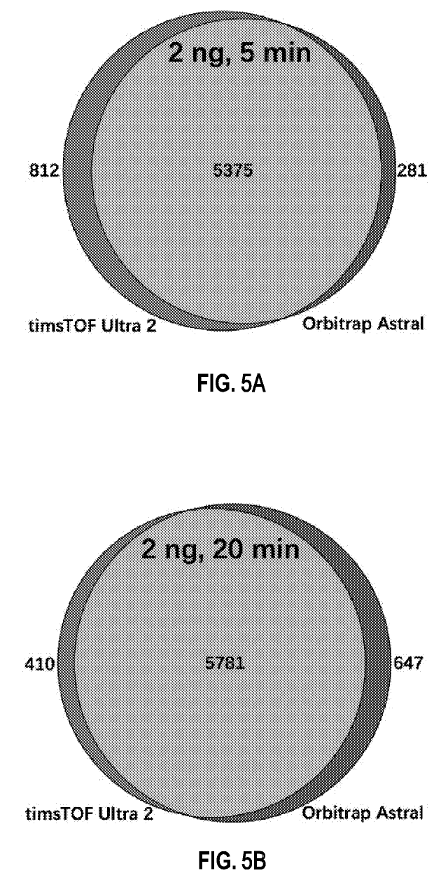

In accordance with the foregoing, some embodiments of the mass spectrometry portion of the system are configured for high-speed or high-throughput operation. For example, in some embodiments, the mass spectrometer is configured to perform rapid or short cycles, or to operate with high acquisition rates, thereby enabling analysis of a large number of samples within a given time period (thereby reducing overall time spent using costly equipment per cycle, and correspondingly reducing the cost of the process). For example, in some cases, the method 100 includes operating the mass spectrometer in short cycles of between about 3 minutes and about 30 minutes (or any suitable subrange thereof, such as between about 6-20 minutes, 7-15 minutes, 8-12 minutes, or any other suitable timeframe) per sample. Indeed, in accordance with some embodiments, a cycle time of about 10 minutes (± 0.5, 1, 1.5, 2, 2.5, or 3 minutes) can be particularly useful for obtaining desirable results at low cost. By way of non-limiting illustration, FIG. 4A shows a variety of gradient times performed in accordance with some embodiments, including a 10-minute cycle.

In some embodiments, the mass spectrometry system is configured to analyze samples at rates corresponding to at least about 50 samples per day, at least about 100 samples per day, at least about 125 samples per day, at least about 250 samples per day, at least about 500 samples per day, or any other suitable throughput, including in workflows configured to approach continuous or near-continuous operation.

In some embodiments, the LC-MS workflow as a whole is configured to operate using one or more standardized configurations. For example, in some embodiments, the LC-MS workflow utilizes one or more predefined chromatographic conditions, column configurations, flow rates, gradient profiles, mass spectrometry acquisition parameters, or other operational settings that are applied across a plurality of samples without substantial modification. In some embodiments, the use of such standardized configurations reduces or eliminates the need for sample-specific optimization of analysis conditions, thereby enabling more efficient processing of multiple samples. In some embodiments, the LC-MS workflow is configured such that the same or substantially similar acquisition method can be applied to samples across a range of input amounts, sample types, or experimental conditions.

In some embodiments, the LC-MS workflow is configured for high-throughput operation. For example, in some embodiments, the LC-MS workflow includes one or more systems configured to analyze a plurality of samples in succession, including by reducing or minimizing idle time between runs. In some embodiments, the LC-MS workflow includes one or more multi-column chromatographic systems, parallelized column configurations, or other arrangements configured to increase utilization of one or more mass spectrometers. In some embodiments, the LC-MS workflow is configured to analyze samples using relatively short chromatographic gradients while maintaining suitable proteome coverage and reproducibility. In some embodiments, such configurations enable analysis of a large number of samples within a given time period, thereby facilitating scalable proteome profiling workflows.

As shown by boxes 150-160 of FIG. 7, some embodiments include one or more of processing data to identify proteome characteristics and generating one or more proteome profiles based on the processed data. In this regard, processing the data can include any suitable computational, statistical, or analytical techniques configured to extract information from the mass spectrometry data. For example, in some embodiments, processing the data includes identifying one or more peptides, proteins, or protein groups represented in the sample, determining one or more characteristics associated with such peptides or proteins (e.g., abundance, presence or absence, modification state, fragmentation patterns, or other properties), or otherwise interpreting the acquired data to generate meaningful proteomic information.

In some embodiments, processing the data includes one or more techniques such as peak detection, feature extraction, spectral matching, database searching, de novo sequencing, quantification, normalization, or any other suitable data processing techniques. In some embodiments, processing the data includes comparing acquired spectra to one or more reference databases (e.g., protein databases, spectral libraries, or other repositories) to identify peptides or proteins present in the sample. In some embodiments, processing the data includes determining relative or absolute abundances of one or more proteins, including via label-free quantification, isotopic labeling approaches, or other quantification techniques. In some embodiments, the data processing includes evaluation of data quality, reproducibility, or confidence metrics associated with identified peptides or proteins.

In some embodiments, processing the data includes one or more automated or semi-automated workflows. For example, in some embodiments, one or more software platforms (e.g., open-source or proprietary tools) are configured to automatically process, analyze, or interpret the acquired mass spectrometry data. In some embodiments, such workflows include one or more of data normalization, calibration using one or more standards (as discussed above), identification of outliers, generation of summary statistics, or other automated processing steps. In some embodiments, the use of automated workflows reduces manual intervention, improves reproducibility, and enables scalable analysis across a plurality of samples.

In some embodiments, generating the proteome profile includes producing one or more outputs representing the proteins present in the sample and one or more characteristics associated therewith. For example, in some embodiments, the proteome profile includes one or more lists, graphs, charts, or any other suitable proteome outputs. In some embodiments, the method 100 includes using the proteome profile for one or more downstream applications, such as comparison across samples, identification of biomarkers, clustering of samples, classification of biological states, or other analytical or diagnostic purposes. In some embodiments, the proteome profile is stored, displayed, transmitted, or otherwise made available for further analysis or interpretation.

According to some embodiments, the method 100 is configured to allow for rapid and systematic proteome profiling of multiple samples (e.g., in quick succession, in multiple locations, or in other manners that allow for cost reduction or increased convenience or efficacy). Accordingly, in some cases, the method is repeated one or more times (with any of the parameters described in connection with the method changing or remaining the same across runs). For example, some embodiments include obtaining a first biological sample and a second biological sample (and any number of additional biological samples, such as a third biological sample, a fourth biological sample, a fifth biological sample, and so on). In some cases, the method includes preparing a first sample preparation from the first biological sample, and preparing a second sample preparation from the second biological sample (and, where applicable, preparing a third sample preparation from the third biological sample, a fourth sample preparation for the fourth biological sample, a fifth sample preparation from the fifth biological sample, and so on). In some embodiments, each sample preparation is introduced into an LC-MS workflow. In some cases, the LC-MS workflow has a single set of operation parameters for all the samples (e.g., a first set of operation parameters is used for multiple sample preparations), whereas in some cases, one or more operation parameters are adjusted between runs (e.g., a first set of operation parameters is used with the first sample preparation, and a second set of operation parameters is used with the second sample preparation, and so on). That said, in some cases, it can be particularly effective to leave all or the majority of the operation parameters the same between runs, so time does not need to be spent adjusting and optimizing between runs. Thus, in some cases, at least 80%, at least 85%, at least 90%, at least 95%, at least 99%, or any other suitable amount of the operation parameters remain the same across samples or between runs. Additional portions of the method can be performed in like manner, with multiple samples being processed identically, similarly, or differently, in accordance with any of the portions of the systems or methods described herein. In some cases, this enables multiple proteomes to be profiled in a cost-effective manner. For example, in some cases, a first set of mass spectrometry data is acquired using a first mass spectrometer, and a second set of mass spectrometry data is acquired using a second mass spectrometer, but the operation parameters remain the same. In some cases, the use of one or more standards (as discussed above) helps enable the use of multiple mass spectrometers or other pieces of equipment without the need for intensive optimization when switching equipment.

Similarly, multiple standards can be used (with one or more samples), such as a first, second, third, fourth, fifth, or any other suitable number of system suitability standards, external standards, internal standards, or other standards.

According to some embodiments, the method includes analyzing a single sample across multiple LC-MS analyses operated under different acquisition conditions (in other words, with different operation parameters). For example, some embodiments include analyzing one sample by first dividing it into multiple subsamples (e.g., 2, 3, 4, 5, 6, or any other suitable number of subsamples). (As illustrated by FIG. 6A, in some embodiments, dividing a 50-ng sample into five 10-ng subsamples can yield almost the same number of proteins for each 10-ng sample; additionally, as illustrated by FIGS. 4A–4B, in some embodiments, decreasing the analysis time from 20 or 30 minutes to 5 or 10 minutes does not substantially impact the number of proteins identified). Accordingly, some embodiments include performing multiple LC-MS analyses of a given sample (e.g., 4), taking a limited amount of time (e.g., 5 minutes each), and using a portion (e.g., 25%) of the total sample for each analysis. In some embodiments, the method includes operating the mass spectrometer under different conditions for each of the multiple analyses, which in some cases allows quantification of more proteins in a given time interval (e.g., 20 minutes) than would be possible with a single 20-min LC-MS analysis that consumes all of the sample at once. In some such cases, each subsample can be prepared and analyzed in any manner as a full sample as described herein (e.g., in accordance with a one-pot preparation technique, together with one or more standards, in accordance with one or more designated operation parameters, or otherwise).

The described systems and methods can be modified in any suitable manner. For instance, in some embodiments, one or more components of the workflow (e.g., the biological sample, the sample preparation, the standards, the liquid chromatography portion, the mass spectrometry portion, or the data processing portion) can be substituted, modified, rearranged, omitted, performed in an overlapping fashion, performed in series, performed in parallel, repeated, or supplemented with additional or alternative components. In some embodiments, one or more components are replaced with functionally equivalent components configured to perform similar roles, including alternative chromatographic systems, alternative mass spectrometry platforms, alternative data acquisition methods, or alternative data processing techniques.

In some embodiments, one or more components are configured to be automated, semi-automated, or manually operated, and may be integrated into one or more systems or platforms. In some embodiments, the systems and methods are configured to operate across different sample types, input amounts, or experimental conditions, including by adjusting one or more parameters while maintaining a generally consistent workflow structure. Similarly, in some cases, one or more components are configured to be selectively activated, deactivated, or adjusted based on one or more conditions or inputs.

In some embodiments, one or more portions of the method 100 can be performed in a different order, performed concurrently, repeated, or omitted, or additional portions can be added.

Additionally, in some embodiments, the described method for performing proteome profiling includes obtaining a biological sample; preparing a sample preparation for proteomic analysis, the sample preparation comprising protein from the biological sample, wherein the sample preparation comprises less than about 100 ng of protein; introducing the sample preparation into a liquid chromatography/mass spectrometry (LC-MS) workflow using a predefined set of acquisition parameters applied without modification across a plurality of samples; acquiring mass spectrometry data using a chromatographic gradient of less than about 30 minutes; processing the mass spectrometry data to identify proteome characteristics; and generating a proteome profile based on the mass spectrometry data after processing, wherein the method further comprises: analyzing a system suitability standard to evaluate performance of the LC-MS workflow; preparing an external standard alongside the biological sample to evaluate sample preparation performance; and combining an internal standard with the sample preparation to normalize the mass spectrometry data.

In some embodiments of the described systems, the sample preparation comprises between about 200 pg and 10 ng of protein. In some embodiments of the described systems, the sample preparation comprises between about 200 pg and 50 ng of protein.

In some embodiments of the described systems, the LC-MS workflow processes at least 100 samples per day.

In some embodiments of the described systems, the acquisition method comprises data-independent acquisition (DIA) or diaPASEF.

In some embodiments, the method includes providing a multi-well plate comprising a plurality of wells, wherein: a first well comprises a system suitability standard, a second well comprises an external standard, and a third well comprises an internal standard; adding a biological sample to the third well; processing contents of the wells using a liquid chromatography/mass spectrometry (LC-MS) workflow; acquiring mass spectrometry data from each of the wells; and evaluating: instrument performance based on the system suitability standard, sample preparation performance based on the external standard, and quantitative normalization based on the internal standard.

In addition to the aforementioned features, the described systems and methods can include any other suitable feature. Indeed, overall, some embodiments of the systems and methods described herein include one or more standardized workflows configured to facilitate consistent, rapid, and inexpensive proteome profiling across a plurality of samples. For example, in some embodiments, one or more of the sample preparation, liquid chromatography, mass spectrometry acquisition, and data processing steps are performed using one or more predefined or consistent parameters across multiple samples. In some embodiments, such standardization reduces variability between runs, simplifies workflow design, and enables reproducible generation of proteome profiles without extensive per-sample optimization. In some embodiments, the systems and methods are configured to operate using relatively small sample inputs, relatively short chromatographic gradients, relatively rapid data acquisition, or relatively fast processing, while still enabling identification and quantification of a substantial portion of the proteome. In some embodiments, the combination of standardized preparation, analysis, and processing steps enables efficient use of instrumentation, reduces consumption of reagents, and facilitates high-throughput proteome profiling across a large number of samples. In some embodiments, the systems and methods enable scalable proteome profiling workflows that can be implemented across different environments, instruments, or laboratories while maintaining consistent performance.

According to some embodiments, a method for performing low-cost proteome profiling is provided. According to some embodiments, a system for performing low-cost proteome profiling is provided. Additional representative embodiments are described below:

The system or method of as described above, further comprising obtaining a biological sample.

The system or method of any of the above, wherein the biological sample is obtained from a clinical subject.

The system or method of any of the above, wherein the biological sample is obtained from the environment.

The system or method of any of the above, further comprising preparing a sample preparation for proteomic analysis.

The system or method of any of the above, further comprising preparing the sample preparation using a one-pot preparation technique.

The system or method of any of the above, the sample preparation comprising less than about 100 ng of protein from the biological sample (e.g., to prevent clogging of a liquid chromatography column due to excess lipids or debris).

The system or method of any of the above, further comprising introducing the sample preparation into a liquid chromatography/mass spectrometry (LC-MS) workflow.

The system or method of any of the above, further comprising acquiring mass spectrometry data from the sample preparation using a mass spectrometer of the LC-MS workflow.

The system or method of any of the above, further comprising using the mass spectrometer for less than about 20 minutes.

The system or method of any of the above, further comprising using the mass spectrometer for less than about 15 minutes.

The system or method of any of the above, further comprising processing the mass spectrometry data to identify proteome characteristics.

The system or method of any of the above, wherein the proteome characteristics include one or more of protein masses, protein types, protein quantities, protein charges, protein abnormalities, or any other proteome characteristics.

The system or method of any of the above, further comprising generating a proteome profile based on the mass spectrometry data after processing.

The system or method of any of the above, further comprising extracting less than about 100 ng of protein from the biological sample.

The system or method of any of the above, further comprising extracting less than about 50 ng of protein from the biological sample.

The system or method of any of the above, further comprising extracting less than about 20 ng of protein from the biological sample.

The system or method of any of the above, further comprising adding the protein from the biological sample to a container.

The system or method of any of the above, wherein the container contains a sample preparation reagent.

The system or method of any of the above, wherein the sample preparation reagent is provided in a stabilized form.

The system or method of any of the above, wherein the stabilized form is a dry form.

The system or method of any of the above, wherein the dry form is an evaporated form.

The system or method of any of the above, wherein the dry for is a freeze-dried form.

The system or method of any of the above, wherein the stabilized form is a chilled form.

The system or method of any of the above, wherein the chilled form is at a temperature of between about 20 ° C. and -120 ° C.

The system or method of any of the above, wherein the chilled form is at a temperature of between about -20 ° C. and -80 ° C.

The system or method of any of the above, wherein the sample preparation reagent comprises at least one of a solvent, an ionization enhancer, a buffer, and a protein preparation reagent.

The system or method of any of the above, further including a second sample preparation reagent (including any of the above).

The system or method of any of the above, further including a third, fourth, fifth, sixth, or any other suitable number of additional sample preparation reagents.

The system or method of any of the above, further comprising reconstituting the sample preparation reagent.

The system or method of any of the above, further comprising incubating the preparation for a period of time.

The system or method of any of the above, wherein the period of time is between about 30 minutes and 120 minutes.

The system or method of any of the above, wherein the period of time is between about 60 minutes and 90 minutes.

The system or method of any of the above, further comprising a standard.

The system or method of any of the above, wherein the standard is provided in a stabilized form (in accordance with any of the above).

The system or method of any of the above, wherein the standard is provided in a container together with the sample preparation reagent.

The system or method of any of the above, wherein the standard comprises a system suitability standard.

The system or method of any of the above, wherein the standard comprises an external standard.

The system or method of any of the above, wherein the standard comprises an internal standard.

The system or method of any of the above, further comprising multiple standards.

The system or method of any of the above, wherein multiple standards are provided in separate containers.

The system or method of any of the above, wherein multiple standards are provided in a single container.

The system or method of any of the above, wherein a standard is preloaded (into a container, either or its own, or together with one or more additional standards or reagents).

The system or method of any of the above, wherein the standard comprises at least one of a protein, a peptide, a labeled peptide, an isotopically labeled compound, or another reference material having a known identity, concentration, or other response characteristic.

The system or method of any of the above, further comprising processing a plurality of biological samples (according to any of the above) in series.

The system or method of any of the above, wherein each of the plurality of biological samples is processed immediately following a prior processing of a prior biological sample to achieve a duty cycle of the LC-MS workflow in excess of approximately 75%.

The system or method of any of the above, wherein each of the plurality of biological samples is introduced into the LC-MS workflow in series at a rate between about 100 and 500 biological samples per day.

The system or method of any of the above, wherein the acquiring the mass spectrometry data comprises evaluating the sample preparation using the mass spectrometer of the LC-MS workflow for less than about 10 minutes (per sample).

The system or method of any of the above, further comprising a group of pre-prepared containers.

The system or method of any of the above, wherein each container of the group of pre-prepared containers contains at least one of the sample preparation reagent and the standard.

The system or method of any of the above, wherein the LC-MS workflow is configured to process the plurality of samples according to a single set of operation parameters.

The system or method of any of the above, wherein the set of operation parameters comprises at least one of: (i) performing a limited-duration liquid chromatography assay configured to take less than 30 minutes; and (ii) performing a limited-duration mass spectrometer assay configured to take less than 30 minutes.

The system or method of any of the above, further comprising evaluating the efficacy of the LC-MS workflow.

The system or method of any of the above, wherein such evaluation comprises determine at least one of: retention time stability, peak shape, peak width, mass accuracy, signal intensity, dynamic range, ion transmission efficiency, and fragmentation efficiency.

The system or method of any of the above, further comprising comparing one or more measured parameters of the system suitability standard to predefined acceptance criteria and accepting or rejecting the mass spectrometry data based thereon.

The system or method of any of the above, wherein the system suitability standard comprises a multi-species proteome mixture having predefined ratios of proteins derived from at least two organisms.

The system or method of any of the above, wherein the system suitability standard comprises human-derived peptides, yeast-derived peptides, and bacteria-derived peptides.

The system or method of any of the above, wherein the system suitability standard comprises a reference peptide mixture.

The system or method of any of the above, wherein the reference peptide mixture includes at least one of a 6x5 peptide mixture and a 7x5 peptide mixture.

The system or method of any of the above, wherein the system suitability standard is analyzed prior to analyzing the biological sample.

The system or method of any of the above, wherein the system suitability standard is analyzed periodically between runs while analyzing a batch of samples.

The system or method of any of the above, further comprising alternating between a plurality of different system suitability standards at predefined intervals.

The system or method of any of the above, wherein he external standard comprises an undigested protein sample that requires processing using the same preparation protocol as the biological sample.

The system or method of any of the above, wherein evaluating the efficacy of the preparation protocol comprises comparing measured peptide outputs from the external standard to expected peptide outputs generated upon successful digestion.

The system or method of any of the above, wherein the external standard comprises a cell lysate having a known protein composition.

The system or method of any of the above, wherein the external standard lacks pre-digested peptides.

The system or method of any of the above, wherein the external standard comprises a peptide having a predefined cleavage site.

The system or method of any of the above, wherein evaluating the efficacy of the preparation protocol comprises determining at least one of: digestion efficiency, peptide yield, recovery efficiency, and reproducibility across samples

The system or method of any of the above, further comprising repeating the preparation protocol when the external standard deviates from expected values by more than a predefined threshold.

The system or method of any of the above, wherein the external standard is prepared in parallel with a plurality of biological samples to identify systematic preparation errors across the plurality of samples.

The system or method of any of the above, wherein providing reference data comprises calculating a ratio between a signal of an analyte and a signal of the internal standard.

The system or method of any of the above, wherein the internal standard is present at a known concentration and is used to normalize signal intensities across multiple samples.

The system or method of any of the above, wherein the internal standard comprises one or more isotopically labeled peptides.

The system or method of any of the above, wherein the internal standard comprises a peptide mixture comprising fewer than a threshold number of peptides to avoid masking analyte signals.

The system or method of any of the above, wherein the internal standard is added prior to sample preparation such that the internal standard undergoes the same preparation steps as the biological sample.

The system or method of any of the above, wherein the internal standard is added after preparation but prior to LC-MS analysis.

The system or method of any of the above, wherein the system suitability standard validates performance of the LC-MS workflow.

The system or method of any of the above, wherein the external standard validates performance of the preparation protocol.

The system or method of any of the above, wherein the internal standard enables normalization of analyte signals.

The system or method of any of the above, wherein the containers comprise wells of a well plate.

The system or method of any of the above, wherein the preloaded standards are arranged across a plurality of wells such that: a first well contains the system suitability standard, a second well contains the external standard, and a third well contains the internal standard.

The system or method of any of the above, wherein at least one well comprises a combination of the system suitability standard, the external standard, and the internal standard.

Any and all of the components in the figures, embodiments, implementations, instances, cases, methods, applications, iterations, and other parts of this disclosure can be combined in any suitable manner. Additionally, any component can be removed, separated from other components, modified with or without modification of like components, or otherwise altered together or separately from anything else disclosed herein.

As used herein, the singular forms “a”, “an”, “the” and other singular references include plural referents, and plural references include the singular, unless the context clearly dictates otherwise. For example, reference to an LC-MS workflow includes reference to one or more LC-MS workflows, and reference to standards includes reference to one or more standards. In addition, where reference is made to a list of elements (e.g., elements a, b, and c), such reference is intended to include any one of the listed elements by itself, any combination of less than all of the listed elements, and/or a combination of all of the listed elements. Moreover, the term “or” by itself is not exclusive (and therefore may be interpreted to mean “and/or”) unless the context clearly dictates otherwise. Similarly, the term “and” by itself is not exclusive (and therefore may be interpreted to mean “and/or”) unless the context clearly dictates otherwise. Furthermore, the terms “including”, “having”, “such as”, “for example”, “e.g.”, and any similar terms are not intended to limit the disclosure, and may be interpreted as being followed by the words “without limitation”.

In addition, as the terms “on”, “disposed on”, “attached to”, “connected to”, “coupled to”, etc. are used herein, one object (e.g., a material, element, structure, member, etc.) can be on, disposed on, attached to, connected to, or otherwise coupled to another object—regardless of whether the one object is directly on, attached, connected, or coupled to the other object, or whether there are one or more intervening objects between the one object and the other object. Also, directions (e.g., “front”, “back”, “on top of”, “below”, “above”, “top”, “bottom”, “side”, “up”, “down”, “under”, “over”, “upper”, “lower”, “lateral”, “right-side”, “left-side”, “base”, etc.), if provided, are relative and provided solely by way of example and for ease of illustration and discussion and not by way of limitation.

The described systems and methods may be embodied in other specific forms without departing from their spirit or essential characteristics. The described embodiments, examples, and illustrations are to be considered in all respects only as illustrative and not restrictive. The scope of the described systems and methods is, therefore, indicated by the appended claims rather than by the foregoing description. All changes that come within the meaning and range of equivalency of the claims are to be embraced within their scope. Moreover, any component and characteristic from any embodiments, examples, and illustrations set forth herein can be combined in any suitable manner with any other components or characteristics from one or more other embodiments, examples, and illustrations described herein.

### Examples

The following examples are provided to illustrate certain implementations of the systems and methods described herein and are not intended to limit the scope of the disclosure. In these examples, parameters were set in accordance with some embodiments of the systems and methods described herein (for example, fixed mass spectrometer acquisition settings were employed across sample inputs and gradient lengths, achieving strong and consistent performance without the need for extensive parameter optimization).

### Example 1 – Proteome Coverage and Reproducibility Across Sample Inputs

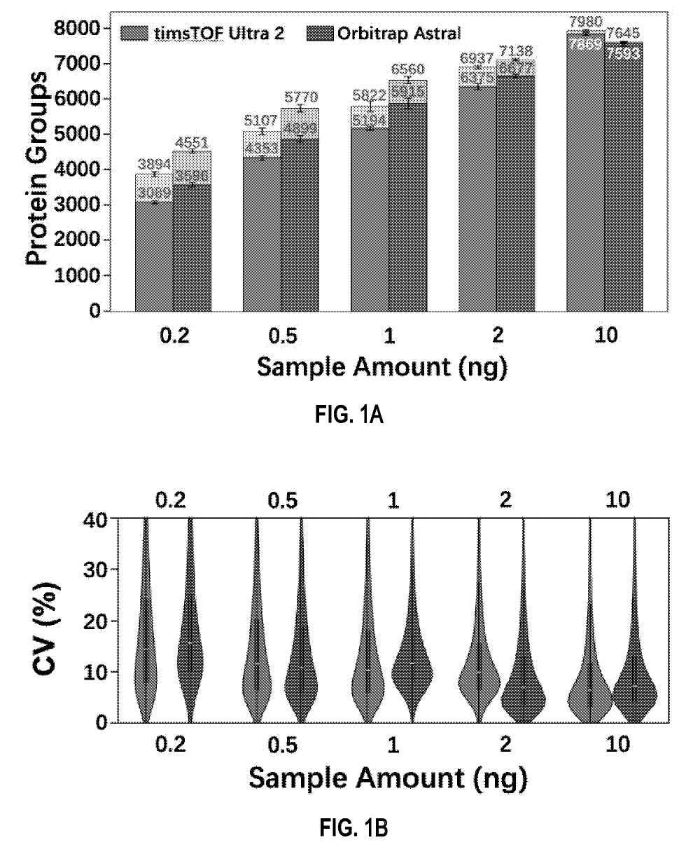

In an example (and in accordance with some embodiments), proteome profiling was performed across a range of sample input amounts, as shown in FIGS. 1A–1B. Sample inputs ranging from about 200 pg to about 10 ng of protein were analyzed using different mass spectrometry platforms. As shown in FIG. 1A, in accordance with some embodiments, proteome coverage increased with sample input, with approximately thousands of protein groups identified even at low input levels and approximately 8,000 protein groups identified at about 10 ng input. The results further demonstrate that proteome coverage begins to approach saturation at low-nanogram input levels (protein coverage began to plateau at the higher end of this range). As shown in FIG. 1B, reproducibility across replicate measurements was, in accordance with some embodiments, maintained across the tested input range, with relatively low coefficients of variation observed, particularly at higher input levels. These results demonstrate that substantial proteome coverage and reproducibility can be achieved using relatively small amounts of proteinaceous material, in at least some embodiments in which the systems and methods described herein are implemented.

### Example 2 – Linearity and Ion Mobility Effects

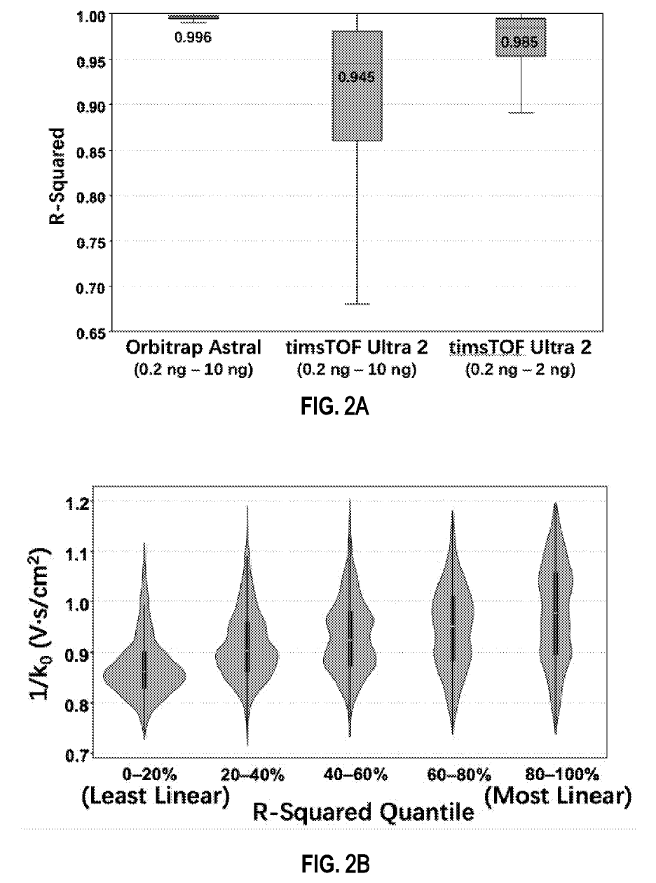

In an example (in accordance with some embodiments), the relationship between peptide signal intensity and sample input was evaluated, as shown in FIGS. 2A–2B. As shown in FIG. 2A, peptide signal intensity exhibited a substantially linear relationship with respect to sample input, with high R² values observed across the tested range, indicating reliable quantitative performance. As shown in FIG. 2B, ion mobility characteristics were evaluated in relation to signal linearity, demonstrating that peptides with different ion mobility properties may exhibit different degrees of linearity. These results indicate that the systems and methods described herein can provide quantitative proteome profiling across a range of sample inputs while accounting for ion mobility effects.

### Example 3 – Cross-Platform Correlation

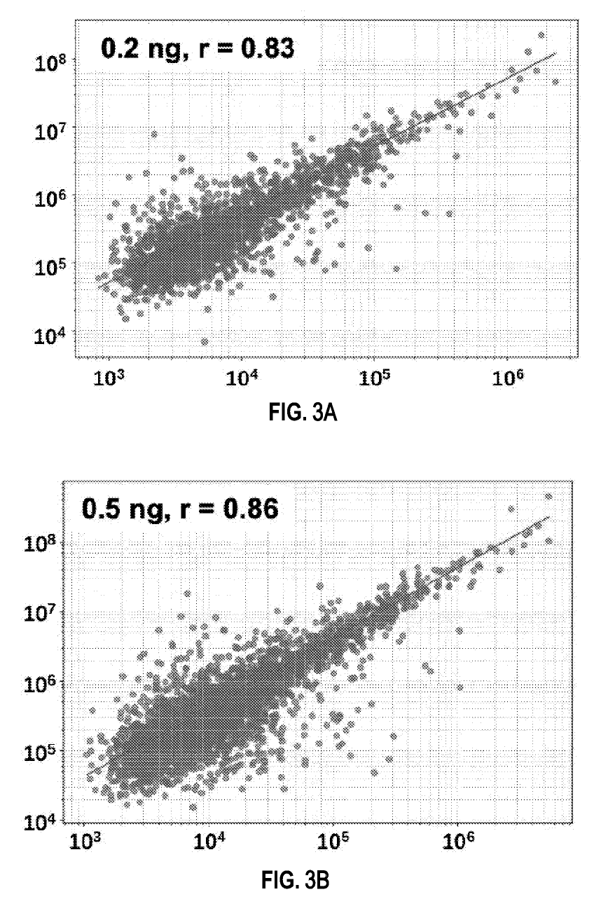

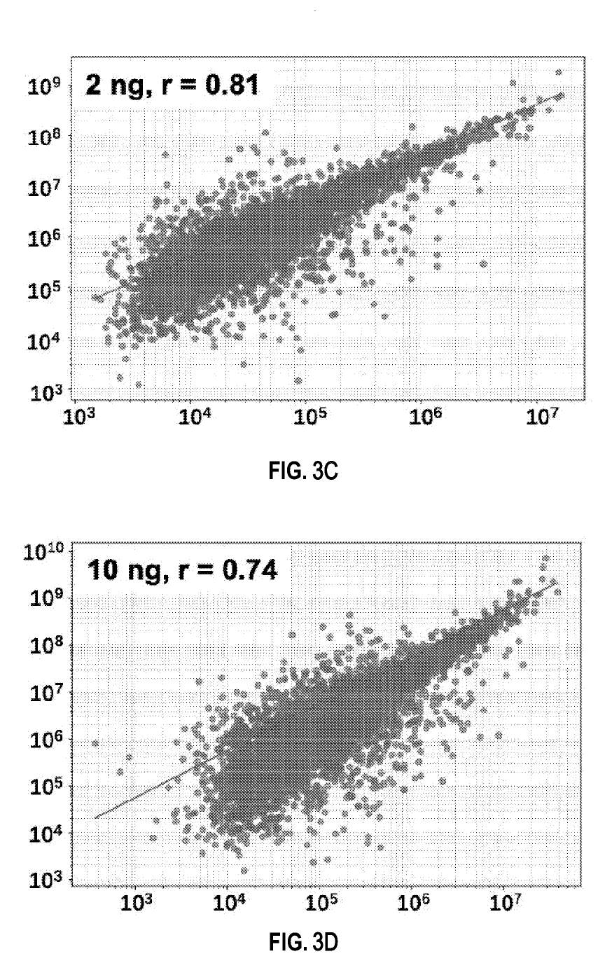

In an example (in accordance with some embodiments), proteome profiling results obtained using different mass spectrometry platforms were compared across multiple sample input levels, as shown in FIGS. 3A–3D. The comparisons included sample inputs of approximately 0.2 ng, 0.5 ng, 2 ng, and 10 ng. The results demonstrate strong correlation between peptide signal intensities across platforms, with high correlation coefficients observed across all tested input levels. These results indicate that the systems and methods described herein can be implemented across different mass spectrometry systems while maintaining consistent and comparable proteomic outputs.

### Example 4 – Effect of Gradient Length on Performance

In an example (in accordance with some embodiments), the effect of chromatographic gradient length on proteome coverage and reproducibility was evaluated, as shown in FIGS. 4A–4B. Gradient durations ranging from about 5 minutes to about 60 minutes were tested. As shown in FIG. 4A, in some embodiments, relatively short gradient lengths (e.g., about 5–20 minutes) provided proteome coverage comparable to longer gradients, with only modest increases observed at longer durations. As shown in FIG. 4B, in accordance with some embodiments, reproducibility remained high across the tested gradient lengths, with relatively low coefficients of variation observed even at shorter gradient durations. These results demonstrate that relatively short chromatographic gradients can be used while maintaining suitable proteome coverage and reproducibility.

### Example 5 – Overlap in Identified Proteins Across Platforms and Conditions

In an example (in accordance with some embodiments), overlap in identified proteins across different mass spectrometry platforms and gradient conditions was evaluated, as shown in FIGS. 5A–5B. The results demonstrate substantial overlap in identified protein groups between platforms under different conditions (e.g., different gradient lengths), indicating that the systems and methods described herein provide consistent identification of proteins across different analytical configurations. These results further support the robustness and cross-platform applicability of the disclosed methods.

### Example 6 – High-Throughput Proteome Profiling Using Multi-Column LC

In an example (in accordance with some embodiments), proteome profiling was performed using a multi-column liquid chromatography system configured for high-throughput operation, as shown in FIGS. 6A–6B. Sample inputs ranging from about 200 pg to about 100 ng were analyzed using a system configured to operate at approximately 126 samples per day (approximately 11.4 minutes per sample). As shown in FIG. 6A, proteome coverage remained substantial across the tested input range, with approximately 6,000 protein groups identified at low input levels and approximately 8,000 protein groups identified at higher input levels. As shown in FIG. 6B, reproducibility remained high across the tested conditions, with low coefficients of variation observed. These results demonstrate that high-throughput proteome profiling can be achieved while maintaining suitable coverage and reproducibility.

### Example 7 – Integrated Low-Cost Proteome Profiling Workflow

In an example (in accordance with some embodiments), the systems and methods described herein were implemented as an integrated workflow combining low-input sample preparation, standardized LC-MS acquisition, and automated data processing, as illustrated collectively in FIGS. 1–6. The workflow utilized relatively small sample inputs, relatively short chromatographic gradients, and standardized acquisition parameters across samples. The resulting data demonstrated that substantial proteome coverage, reproducibility, and quantitative performance can be achieved under these conditions. In some implementations, the workflow enables analysis of a large number of samples within a given time period, thereby supporting scalable and efficient proteome profiling.

### Example 8 – Method Workflow for Proteome Profiling

In an example (in accordance with some embodiments), a method for performing proteome profiling was implemented as a structured workflow, as illustrated in FIG. 7. The method included obtaining a biological sample, preparing a sample preparation for proteomic analysis, introducing the sample preparation into an LC-MS workflow, acquiring mass spectrometry data, processing the data to identify proteome characteristics, and generating a proteome profile based on the processed data. In some implementations, one or more of the steps were performed using standardized parameters across multiple samples, thereby enabling consistent and reproducible proteome profiling. In some implementations, the workflow was configured to operate using relatively small sample inputs and relatively short analysis times, while still providing substantial proteome coverage.

### Example 9 – Use of System Suitability, External, and Internal Standards

In an example (in accordance with some embodiments), proteome profiling was performed using one or more standards incorporated into the workflow, including system suitability standards, external standards, and internal standards. The system suitability standards were used to evaluate performance of one or more components of the LC-MS workflow, including chromatographic separation and mass spectrometry analysis. The external standards were prepared alongside biological samples and were used to evaluate effectiveness of sample preparation processes. The internal standards were incorporated into the sample preparations and were used to normalize signal intensities and improve quantitative accuracy. In some implementations, the combined use of these standards enabled reliable proteome profiling even under conditions involving relatively small sample inputs and standardized acquisition parameters.

### Example 10 – Preloaded Standards in Multi-Well Plate Format

In an example (in accordance with some embodiments), one or more standards were provided in a preloaded format using a multi-well plate. The multi-well plate included a plurality of wells containing one or more preloaded standards, including system suitability standards, external standards, and internal standards. In some implementations, one or more wells were configured to receive biological samples, while other wells were configured to contain standards for quality control and calibration. In some implementations, the preloaded standards were arranged across the wells in a predetermined configuration to facilitate standardized workflows and high-throughput analysis. The use of such preloaded plates reduced manual preparation steps and improved reproducibility across multiple samples.

Using embodiments of the systems and methods described herein, in-depth global proteome profiling of a wide variety of sample types may be feasible for between about $5 and $50 per sample (e.g., approximately $10 in direct costs per sample, in some cases) resulting in substantial savings over competing proteome profiling (which in some cases can cost thousands of dollars or more). In this regard, conventional mass spectrometry-based proteomics remains technically demanding and prohibitively expensive in many cases, with per-sample costs often ranging from $200 to over $1,000, whereas the present systems and methods can enable in-depth, quantitative global proteome profiles at dramatically reduced cost. In some embodiments, such cost reductions are achieved through a combination of simplified, low-cost sample preparation (e.g., one-pot or one-step workflows suitable for low-nanogram inputs), use of standardized LC-MS acquisition parameters that reduce or eliminate per-sample optimization, and high-throughput operation (e.g., short analysis times on the order of about 10 minutes per sample and high duty cycle configurations). In some implementations, the systems further leverage high-performance mass spectrometers and automated data processing workflows to maintain substantial proteome coverage and reproducibility while reducing instrument time, reagent consumption, and labor, thereby enabling scalable and cost-effective proteome profiling across a large number of samples. Thus, it is worth emphasizing that the cost savings can be dramatic, even to the point of potentially reducing costs for proteome profiling by several orders of magnitude.

### Example 11 – Liquid Chromatography Workflow of some Embodiments

For experiments corresponding to FIGS. 1–5, an Ultimate 3000 RSLCnano LC system (Thermo Fisher, Waltham, MA) was operated at a flow rate of 680 nL/min. The flow was split such that mobile phase passed through the analytical column at ~70 nL/min, while the remaining flow served as the loading pump for the trap-and-elute sample injection. The analytical column was 20 cm long, 30 μm inner diameter, and was packed in-house with 1.9 μm Dr. Maisch C18 media. Mobile phase solvents and wash solutions were purchased from Honeywell (Charlotte, NC). Pierce HeLa peptide digest and formic acid ampules were purchased from Thermo Fisher. A 10-port Nanovolume valve was purchased from VICI (Houston, TX). The 20-min gradients from solvent A (0.1% formic acid in water) to solvent B (0.1% formic acid in acetonitrile) were as follows: After 1 μL of sample was loaded onto the trap column, the gradient was increased from 1% B to 5% B over 3 minutes, then to 25% B over 20 minutes, to 45% B over 4 minutes, to 80% B over 2 minutes for washing, where it was held for 5 minutes before decreasing to 25% B over 3 minutes and then washing at 80% B for another 5 minutes. The 80% B was then decreased to 1% where it remained for 11 min to recondition the column. For other gradient lengths, the active gradient from 5–25% B was adjusted while holding other portions of the gradient the same. For data corresponding to FIGS. 6A–6B when a 2-column system was used, the LC columns were purchased from MicrOmics Technologies, LLC (Spanish Fork, UT). The lengths remained 20 cm, but the bore was 50 μm and the emitter was integrated with the column. The flow rate was ~100 nL/min. The total cycle time was 11.4 min, corresponding to 126 samples per day, and the duty cycle (active gradient/cycle time) was close to 100%.

### Example 12 – Mass Spectrometry Workflow of some Embodiments

The data acquisition method for samples analyzed on the Bruker timsTOF Ultra 2 mass spectrometer in this example was as follows: The LC system was interfaced with a home-etched 10 μm emitter inserted into a CaptiveSpray ESI source, with 1500 V electrospray potential. Full MS data were acquired in the range of m/z 100–1700 and 1.45–0.64 1/K0 [V·s/cm2] in diaPASEF mode. Fifteen DIA windows spanned 400–1000 m/z and were acquired with trapping and ramp times of 150 ms (100% duty cycle). The estimated cycle time was 0.94 s. The collision energy was ramped as a function of increasing mobility starting from 20 eV at 0.6 1/k0 to 63 eV at 1.6 1/k0. No denoising mode was applied for diaPASEF data reduction. With the exception of the data collected with the 2-column nanoLC system corresponding to FIGS. 6A–6B, other samples that were acquired on the Orbitrap Astral mass spectrometer used the following method: A single MS1 scan using the orbitrap detector at 240,000 resolution (at 200 m/z), with the precursor range of m/z 380–980, RF lens setting of 45%, maximum ion injection time set to 100 ms, and the automatic gain control (AGC) target set to 500%. MS2 spectra were simultaneously acquired with the Astral detector, with the following settings: 99 scan events with 4 Da isolation window, 25% HCD collision energy, 45% RF lens, 800% AGC target, 10 ms maximum injection time, and a precursor mass range of 400–800 m/z with window placement optimization. The LC system, column and emitter was the same as for the experiments performed on Bruker timsTOF instrument. The Nanospray Flex ion source was used with an electrospray voltage set to 2200 V. The ion transfer tube temperature was set to 200 ° C. For data acquired using the dual-column nanoLC system (corresponding to FIGS. 6A–6B), the Orbitrap Astral equipped with the FAIMS Pro interface was used. The compensation voltage on the FAIMS interface was fixed at –45 V. The carrier gas flow was 3.6 L/min. Other settings were as described above except for the following settings. MS1 scan range was set to m/z 400–800. The isolation window and injection time was set to 8 Da and 12 ms for sub-2 ng samples, while 4 Da and 6 ms was used for higher-input samples with window placement optimization set to off.

### Example 13 – Data Analysis Workflow of some Embodiments

Raw files (Bruker .d and Thermo .raw) were analyzed directly with DIA-NN 2.0.2 (53). For Bruker timsTOF Ultra data, both mass accuracy and MS1 accuracy were set to 15. For Orbitrap Astral data, mass accuracy was set to 10, and MS1 accuracy was set to 4. Trypsin/P protease specificity allowed up to 1 missed cleavage. Carbamidomethylation of cysteines was a fixed modification, N-term M excision, and precursor charge range of 1+ to 7+. Other precursor and fragment parameters were set to match the MS acquisition methods. Postprocessing was performed using python. Data presented in FIGS. 6A–6B were identified and quantified using DIA-NN (v2.0) and Spectronaut (v19.9), with the human UniProt protein database downloaded on July 16, 2025. For the DIA-NN analysis, a custom experimental spectral library was used. This library was constructed from five 100 ng HeLa digest standard runs acquired on the same instrument, using DIA-NN’s library-free mode with deep learning-based identification enabled. Trypsin/P was specified as the digestion enzyme, allowing up to two missed cleavages. Up to five variable modifications were considered, including N-terminal methionine excision, carbamidomethylation (fixed), methionine oxidation, and N-terminal acetylation. The search was performed with a peptide length range of 7–52 amino acids, precursor charge states of 1–4, a precursor m/z range of 400–800, and a fragment ion m/z range of 150–2000. MS1 and MS2 mass accuracies were set to 0 ppm, and precursor FDR was controlled at 1%. Cross-validated neural networks were used for scoring, and cross-run normalization was disabled. The same parameters were applied in the subsequent library-based search, where raw files acquired on the Orbitrap Astral were analyzed together in a single DIA-NN project. For the Spectronaut analysis, the directDIA+ workflow was used to analyze raw files together in a single project. Trypsin/Lys-C was specified as the digestion enzyme, with a peptide length range of 7–52 amino acids and up to two missed cleavages. A maximum of five modifications was allowed per peptide, including fixed carbamidomethylation and variable modifications such as protein N-terminal acetylation and methionine oxidation. The false discovery rate for PSMs, peptides, and protein groups was controlled at 1%. Quantification was performed at the MS1 level, and cross-run normalization was disabled.

### Example 14 – Window Size

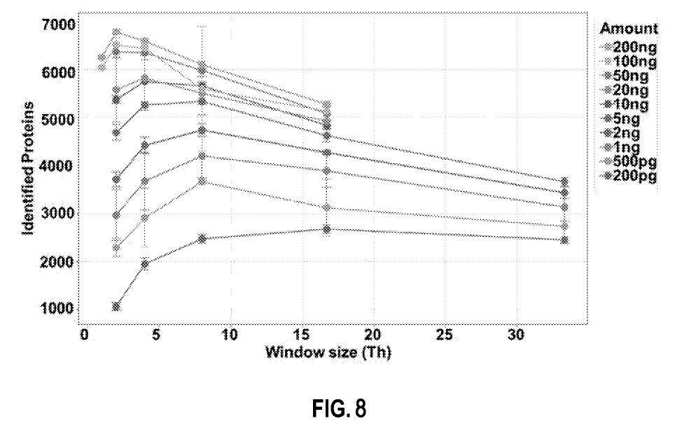

Generally speaking, many competing methods by default include intensive optimization of MS acquisition parameters for a given project (e.g., for each individual run), which costs a lot of time and energy. However, in accordance with some embodiments, by analyzing samples within a given range of amounts and with a limited number of LC settings and MS instrument models and operation parameters, we can select a generally applicable set of operation parameters (e.g., instrument settings) and save a lot of time. By way of non-limiting illustration, FIG. 8 shows that in one example (in accordance with some embodiments), a number of separations were performed that took 2.88 minutes each (~500 samples per day). In this example, for sample amounts from 500 pg to 10 ng, the optimal window size in the MS acquisition was 8 Th (Thomsons). Also, for larger and smaller sample amounts, in this example, this setting was not too far from the optimum. Further, in this example, just 3 window sizes provide optimal settings across sample amounts spanning a 1000x range from 200 pg to 200 ng. Accordingly, in some embodiments, a window size of between 1 and 20 Th is selected, or any suitable range therein (e.g., between 3 and 17 Th, between 5 and 10 Th, 8 Th, or any other suitable size).

Some embodiments of the method 100 include implementing any of the workflows, operation parameters, protocols, or other portions of any of the examples, alone or in combination with any other examples or embodiments, and in some cases plus-or-minus up to 5%, 10%, 15%, 20%, 25%, 30%, or 50% of the stated values therein. By way of non-limiting illustration, where a ramp time of 150 ms is stated, operation parameters can include a ramp time of 150 ms ± 15 ms (10% of 150 ms), or any other suitable adjustment to provide a reasonable operation parameter range of the values presented in the example in question.

What is claimed is:

1. A method for performing low-cost proteome profiling, the method comprising:
obtaining a biological sample;
preparing a sample preparation for proteomic analysis using a one-pot preparation technique, the sample preparation comprising less than about 100 ng of protein from the biological sample;
introducing the sample preparation into a liquid chromatography/mass spectrometry (LC-MS) workflow;
acquiring mass spectrometry data from the sample preparation using a mass spectrometer of the LC-MS workflow for less than about 20 minutes;
processing the mass spectrometry data to identify proteome characteristics; and
generating a proteome profile based on the mass spectrometry data after processing.

2. The method of claim 1, wherein the one-pot preparation technique comprises adding the protein from the biological sample to a container housing a sample preparation reagent in a stabilized form.

3. The method of claim 2, wherein the sample preparation reagent comprises a standard comprising at least one of a preloaded internal standard, a preloaded external standard, and a preloaded system suitability standard.

4. The method of claim 3, wherein the standard comprises at least one of a protein, a peptide, a labeled peptide, an isotopically labeled compound, and another reference material having a known response characteristic.

5. The method of claim 1, wherein the method further includes processing the sample preparation with a chromatography system of the LC-MS workflow for less than about 30 minutes.

6. The method of claim 1, wherein the LC-MS workflow comprises a multi-column chromatography system.

7. The method of claim 1, wherein the acquiring mass spectrometry data comprises evaluating the sample preparation using the mass spectrometer of the LC-MS workflow for less than 10 minutes.

8. The method of claim 1, further comprising analyzing a system suitability standard using the LC-MS workflow alongside the sample preparation to evaluate an efficacy of the LC-MS workflow.

9. The method of claim 1, further comprising preparing an external standard alongside preparing the biological sample to evaluate an efficacy of a preparation protocol.

10. The method of claim 1, further comprising combining the sample preparation with an internal standard to provide reference data when acquiring mass spectrometry data.

11. The method of claim 1, further comprising using a standard as a control configured to produce standard data as part of the mass spectrometry data, wherein the processing the mass spectrometry data to identify proteome characteristics comprises using software configured to automatically identify the standard data and compare an additional portion of the mass spectrometry data to the standard data.

12. A method for performing low-cost proteome profiling, the method comprising:
obtaining a first biological sample and a second biological sample;
preparing a first sample preparation from less than about 100 ng protein of the first biological sample;
preparing a second sample preparation from less than about 100 ng protein of the second biological sample;
introducing the first sample preparation into a liquid chromatography/mass spectrometry (LC-MS) workflow with a first set of operation parameters;
acquiring a first set of mass spectrometry data based on the first sample preparation using the first set of operation parameters;
generating a first proteome profile based on the first set of mass spectrometry data;
introducing the second sample preparation into the LC-MS workflow with the first set of operation parameters;
acquiring a second set of mass spectrometry data based on the second sample preparation using the first set of operation parameters; and
generating a second proteome profile based on the second set of mass spectrometry data.

13. The method of claim 12, wherein the acquiring a first set of mass spectrometry data is performed using a first mass spectrometer, and wherein the acquiring a second set of mass spectrometry data is performed using a second mass spectrometer.

14. The method of claim 12, wherein the first set of operation parameters comprises at least one of: (i) performing a limited-duration liquid chromatography assay configured to take less than 30 minutes; and (ii) performing a limited-duration mass spectrometer assay configured to take less than 30 minutes.

15. The method of claim 12, wherein the preparing the first sample preparation from the first biological sample comprises including less than 100 ng of protein in the sample preparation.

16. The method of claim 12, wherein the first set of operation parameters comprises combining at least one of a first set of internal standards and a second set of internal standards with a sample preparation to be tested.

17. A group of pre-prepared containers for performing proteome profiling, the group of pre-prepared containers comprising:
a first container containing:
a sample preparation reagent in a stabilized form; and
a standard in a stabilized form, the standard comprising at least one of: (i) a system suitability standard; (ii) an external standard; and (iii) an internal standard;
wherein the first container is configured to receive a biological sample and to be processed in a liquid chromatography/mass spectrometry (LC-MS) workflow.

18. The group of pre-prepared containers of claim 17, wherein the standard comprises at least one of a protein, a peptide, a labeled peptide, an isotopically labeled compound, and another reference material having a known response characteristic.

19. The group of pre-prepared containers of claim 18, wherein the first container is configured to receive less than about 100 ng of protein of the biological sample, such that the LC-MS workflow is configured to be completed in less than about 30 minutes.

20. The group of pre-prepared containers of claim 17, wherein the group of pre-prepared containers comprises a second container comprising the system suitability standard, the external standard, and the internal standard.
